(19) United States
(12) Patent Application Publication
(10) Pub. No.: US 2023/0008906 A1
(43) Pub. Date: Jan. 12, 2023

(54) A METHOD AND SYSTEM FOR MONITORING AND ANALYSING COUGH

(71) Applicant: VITALOGRAPH (IRELAND) LTD., Ennis, Co., Clare (IE)

(72) Inventors: Terence Taylor, Ennis, Co. Clare (IE); Yaniv Zigel, Ennis, Co. Clare (IE); Frank Keane, Ennis, Co. Clare (IE); Marcus Garbe, Ennis, Co. Clare (IE)

(21) Appl. No.: 17/777,722

(22) PCT Filed: Nov. 20, 2020
(86) PCT No.: PCT/EP2020/082977

§ 371 Date: May 18, 2022

(30) Foreign Application Priority Data
Nov. 20, 2019 (EP) 19210438.8

(51) Int. Cl. A61B 5/08; A61B 5/113

(52) U.S. Cl. CPC A61B 5/0823; A61B 5/113; A61B 5/72

(57) ABSTRACT

The method and system for monitoring cough comprises receiving audio signals or audio recordings, where said signals or audio recordings comprises one or more of silent segments, cough sound segments, speech segments and extraneous noise. The processing of said received sound signals or sound recordings comprise one or more of removing one or more speech components from speech segments to render the speech unintelligible and clipping said silent segments, wherein one or more speech components include vowel sounds. Further processing of said received audio signals or audio recordings further comprises compressing said audio signals or audio recordings. In the alternative, processing of audio signals or audio recordings comprises compressing a resultant signal after said removal of one or more speech components and/or clipping of silent segments from said audio signals.

### FIELD

The present disclosure relates to a method and system for monitoring cough. More specifically, the present disclosure relates to efficient recording and analysis of the cough of a subject individual.

### BACKGROUND

The objective monitoring of cough for extended periods of time has long been recognized as an important step towards a better understanding of this symptom, and a better management of patients with chronic cough symptoms and to provide objective measurements for the investigation and treatment of chronic cough, and conducting clinical research aimed at finding therapies for those patients whose cough is resistant to treatment.

Some cough monitoring systems use a video recording of patients and then a specialist analyses the recorded video data. The specialist has to analyse this data can take up to 2 weeks to analyse 24 hours of video data. This video based approach is slow and inefficient.

The monitoring of cough symptoms can also be obtained using a microphone. In such a case, the subject individual may easily install such a device on himself/herself and carry out normal day to day activities. The number of coughs conceptually may be counted automatically but such a method has proven to have unsatisfactory accuracy, requiring manual analysis to achieve the required level of accuracy. Manually analysing a recording length of 24 hours may take up to 60 hours which is impractical.

Also, data privacy is another issue while using a microphone as it may record private conversations not limited to a subject's own speech along with the cough sounds. Subjects in clinical trials may give permission for such type of recording, but this will not extend to data subjects not part of the clinical trial or recordings made outside of clinical trials whose voices and conversations may also be recorded. Hence, this type of monitoring may be in contravention to privacy laws in various countries.

A paper publication by Liaqat Daniyal et al ‘A method for preserving privacy during audio recordings by filtering speech’, 2017 IEEE Life Sciences Conference, discloses continuous monitoring of sensor data from wearable devices, such as a smart watch. Liaqat does not indicate whether the said approach taken impedes relevant information from cough audio events. In many cases, cough events contain a “voiced” phase usually at the end of the cough event. This event is imperative for assessing cough. It is essential that the voiced phase of the cough event is not interfered with. Liaqat also fails to report any measures of efficacy specifically relating to speech intelligibility, only its effect on cough detection. Other patent publications in art include CN 108 294 756 and WO 2013 040485 suffer from similar problems to Liaqat.

Therefore, there is a need for a method and system to efficiently monitor cough symptoms for extended periods of time, by recording the symptoms using microphones without affecting the privacy of the subject individual or of individuals surrounding said subject individual and only requiring a fraction of the recorded time for a semi-skilled person to monitor and analyse the cough symptoms of a subject individual.

### SUMMARY

The present invention relates to a method and system for monitoring cough, as set out in the appended claims. More specifically, the present invention relates to efficient recording and analysis of the cough of a subject individual.

The system for monitoring cough comprises a cough monitor. The cough monitor comprises a processor, a microphone module operatively coupled to the processor and a memory operatively coupled to the processor. The processor is configured to receive signals from said microphone module, said signals comprising audio, said audio comprising one or more of silent segments, cough sound segments and speech and/or extraneous noise.

In one embodiment there is provided a cough monitor for a subject, comprising:
a processor;
a microphone module, having a first microphone and a second microphone, operatively coupled to the processor;
a memory operatively coupled to the processor;
said processor configured to:
   receive signals from said first microphone and second microphone, said signals comprising audio, wherein the audio comprises cough sound segments and speech segments to define a cough audio event;
   process said received signals from said microphone module by removing one or more speech components from speech segments to render the speech unintelligible, such that only speech utterances are removed from the cough audio event; and
   store said processed signals in said memory.

Cough events contain a “voiced” phase usually at the end of the cough event. This event is imperative for assessing cough. The present invention specifically ensures that only speech utterances are removed from the audio while not affecting the voiced phase of cough events. The prior art fails to process any measures of efficacy specifically relating to speech intelligibility, only its effect on cough detection.

In one embodiment specific audio features are extracted to detect speech utterances from an audio signal received from said first microphone.

In one embodiment the specific audio features extracted comprises a measure of periodicity in the audio signal relating to the vibration of the vocal folds within a specific frequency range using a custom autocorrelation function.

In one embodiment said processing comprises the step of using values of surrounding audio frames to determine a voiced threshold value for detecting speech over a specific frequency range.

In one embodiment the specific frequency range is between 45-500 Hz.

In one embodiment processing said signals comprises measuring the changes in acoustic energy over time using an energy ratio to discriminate between speech utterances and cough events.
In one embodiment the energy ratio comprises a measure of the ratio of acoustic energy between the first microphone and the second microphone to discriminate between cough events and third party speech.

Further, the processor is configured to process said received signals from said microphone module and store said processed signals in said memory. Processing said received signals comprise one or more of removing one or more speech components from speech segments to render the speech unintelligible and clipping said silent segments where one or more speech components include vowel sounds, and/or removing extraneous non-cough noise. Also, processing said received signals further comprises compressing said signals comprising said audio, or compressing a resultant signal after said removal of one or more speech components and/or clipping of silent segments from said signals comprising audio.

In one embodiment the microphone module comprises a first microphone and a second microphone each configured with a separate channel.

It will be appreciated that by clipping the silent audio segments from the audio recordings and compressing the remaining audio segments significantly reduces the play time a semi-skilled personnel needs to review/analyse for obtaining the objective cough information of the subject/patient. Further, since the speech in the remaining segments would be rendered unintelligible, hence, the privacy of the subject would be maintained.

The method and system for recording uses a microphone for monitoring a cough for extended periods without affecting the privacy of the subject individual or of individuals surrounding said subject individual and only requires a fraction of recorded time for a semi-skilled person to monitor cough of a subject/patient.

The cough monitor further comprises an accelerometer operatively coupled to said processor to obtain the severity of cough from said accelerometer readings, wherein said accelerometer is mechanically coupled to the chest of the subject. The cough monitor further comprising a wireless transceiver for transmitting said processed signals to a server.

In one embodiment the cough monitor further comprises gyroscope operatively coupled to said processor. In one embodiment the gyroscope is configured to determine the position of a person relative to ground when a cough is measured.

The microphone module comprises an air microphone configured to be attached to the lapel and a contact microphone configured to be attached to the chest of the subject. The air microphone and contact microphone are connected to the cough monitor via a single connection port. In an embodiment, the microphone module comprises an air microphone built into the cough monitor and a contact microphone built into the cough monitor, said cough monitor and said contact microphone configured to be attached to the chest of the subject using a biodegradable adhesive.

In an embodiment, the processor is configured to store the processed signals in a selected format selected from a set of predetermined formats. In an embodiment, memory comprises a solid state drive, a removable secure digital card or memory, and/or memory encrypted with Advanced Encryption Standard 256 bit, as an example.

In an embodiment, the cough monitor switches on when a removable secure digital card is inserted in a secure digital card slot of the cough monitor and switches off in absence thereof.

In an embodiment, the processor is configured to detect one or more fault conditions. The fault condition may comprise one or more of the following: low battery, battery door removal, faulty sensors, short circuit across sensors, open circuit across sensors, insufficient memory, memory absent, and/or clock reset.

The cough monitor further comprising a user interface to allow the subject to mute said air microphone, or indicate waking time, or indicate sleeping time, or indicate medication dosing time or other events relevant to research or condition.

In an embodiment, the system comprises a server, where the server is configured to receive one or more audio recordings from a cough monitor via a network. The server may also receive one or more audio recordings physically on a secure digital card. The audio recordings comprising one or more of silent segments, cough sound segments and speech segments. The server is configured to process said audio recordings where processing of said received audio recordings comprise one or more of removing one or more speech components from speech segments to render the speech unintelligible and clipping said silent segments where one or more speech components include vowel sounds. Also, processing said received signals further comprises compressing said audio recordings comprising said audio, or compressing a resultant audio recordings after said removal of one or more speech components and/or clipping of silent segments from said audio recordings comprising audio.

The method for cough monitoring comprises receiving signals from a microphone module, said signals comprising audio, said audio comprising one or more of silent segments, cough sound segments and speech segments, processing said received signals from said microphone module and storing said processed signals in memory. The processing of said received signals comprise of removing one or more speech components from speech segments to render the speech unintelligible and clipping said silent segments.

Further, processing of said received signals comprises compressing said signals comprising said audio or compressing a resultant signal after said removal of one or more speech components and/or clipping of silent segments from said signals comprising audio, wherein one or more speech components include vowel sounds.

The method further comprises transmitting said processed signals to a server via a wireless transceiver. The method further comprises detection of one or more fault conditions comprising low battery, battery door removal, faulty sensors, short circuit across sensors, open circuit across sensors, insufficient memory, memory absent, and/or clock reset.

In one embodiment there is provided the step of monitoring the status of the module by determining the status of energy harvesting parameters during use. For example movement of the subject can generate an electric current to provide power for the sensor which needs to be stored (in a capacitor or battery), the charge state of such a system can also be monitored.

In another embodiment there is provided cough monitor for a subject, comprising:
a processor;
a microphone module, having a first microphone and a second microphone, operatively coupled to the processor;
a memory operatively coupled to the processor;
said processor configured to:
   receive signals from said first microphone and second microphone, said signals comprising audio, wherein the audio comprises cough sound segments and speech segments to define a cough audio event;
   process said received signals from said microphone module by synthesising one or more speech components from speech segments to render the speech unintelligible, such that only speech utterances are synthesised from the cough audio event; and
   store said processed signals in said memory.

### BRIEF DESCRIPTION OF THE DRAWINGS

The invention will be more clearly understood from the following description of an embodiment thereof, given by way of example only, with reference to the accompanying drawings, in which:—

FIG. 1 exemplarily illustrates a flowchart of the method for cough monitoring;

FIG. 2 exemplarily illustrates a block diagram of the cough monitor device; and

FIGS. 3-6 illustrates a number of speech and cough audio signals outputted by the algorithm according to an embodiment of the present invention.

### DETAILED DESCRIPTION OF THE DRAWINGS

The present invention relates to a method and system for monitoring a cough. More specifically, the present invention relates to efficiently recording and analysing a cough.

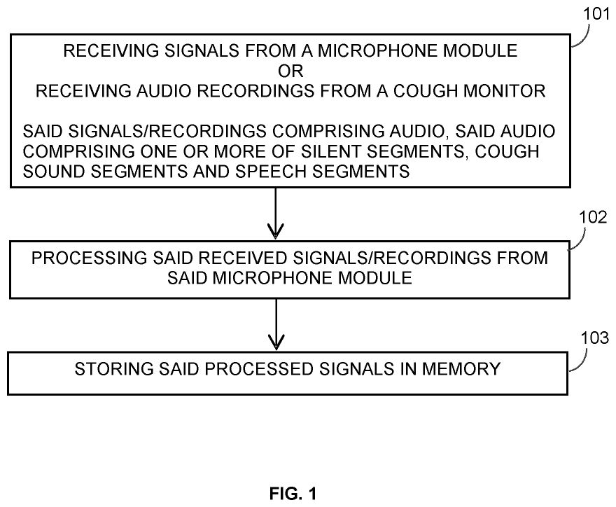

FIG. 1 exemplarily illustrates a flowchart of the method for cough monitoring. The method for cough monitoring comprises receiving 101 signals from a microphone module, said signals comprising audio. In one embodiment the microphone module comprises a first microphone and a second microphone each configured with a separate channel. Alternatively, audio recordings may be received 101 via a network or physically on a removable memory device such as a secure digital card. The audio signals or recordings comprise of one or more of silent segments, cough sound segments and speech segments and/or extraneous noise. The audio signals or recordings are processed 102 and the processed signals or recordings stored 103 in memory thereafter. The processing of said received sound signals or sound recordings comprise of removing one or more speech components from speech segments to render the speech unintelligible and clipping said silent segments, wherein one or more speech components include vowel sounds.

Further, processing of said received audio signals or audio recordings comprises compressing said audio signals or audio recordings. In the alternative, processing of audio signals or audio recordings comprises compressing a resultant signal after said removal of one or more speech components and/or clipping of silent segments from said signals comprising audio.

In an embodiment, the processing of the audio signals is carried out by a cough monitoring device and said processed signals are then transmitted to a server via a wireless network. The method further comprises detection of one or more fault conditions comprising low battery, battery door removal, faulty sensors, short circuit across sensors, open circuit across sensors, insufficient memory, memory absent, and/or clock reset, by the cough monitoring device. It will be appreciated that in the context of the present invention the microphone can be interpreted as a sensor.

The processed audio signals or audio recordings are then reviewed by semi-skilled personnel. The personnel thereafter identifies the cough sounds by listening to said processed audio signals or audio recordings. A person skilled in the art would appreciate that cough sounds are generally divided into three phases namely the explosive phase, the intermediate phase and the voiced phase. The personnel tags the explosive phase of each cough sound to finally generate cough data for a subject. A person skilled in the art would appreciate that various cross checks or quality assurance audits or checks may be carried out to rule out human error in identification of coughs. For example, the timeline of each recording is maintained across the process, with cough tags and events marked/timestamped. The cough tags and events can be ascertained from measurements obtained from the accelerometer or a gyroscope. In the context of the present invention coughs tagged by the skilled person are events. Other events can be from the subject pressing event marker buttons. Measurements from the accelerometer/gyroscope can indicate severity of cough and support skilled person in identify a sound signal as a cough. These events can be marked as a timed event.

Also, a person skilled in the art would appreciate that by clipping the silent audio segments from the audio recordings and compressing the remaining audio segments significantly reduces the play time a semi-skilled personnel needs to review/analyse for obtaining the objective cough information of the subject/patient. Further, since the speech in the remaining segments would be rendered unintelligible, hence, the privacy of the subject is maintained.

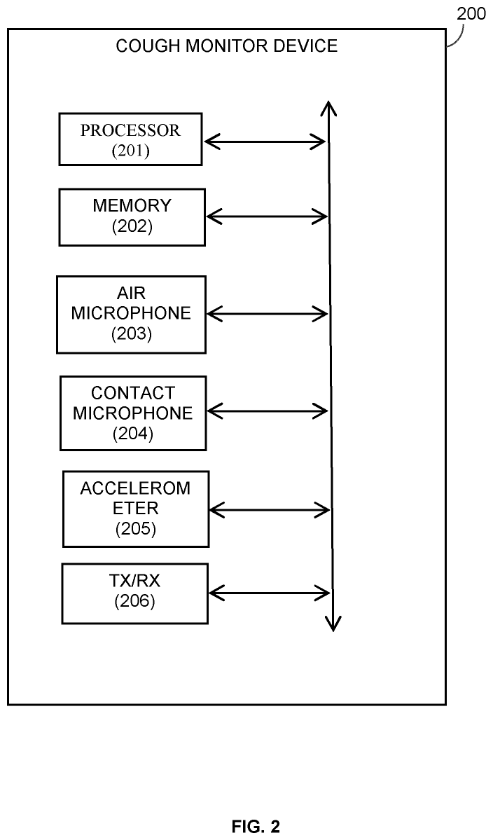

The system for monitoring cough comprises a cough monitor 200 of a subject and FIG. 2 exemplarily illustrates a block diagram of the cough monitor device 200.

The cough monitor comprises a processor 201, a microphone module operatively coupled to the processor and a memory 202 operatively coupled to the processor. The processor 201 is configured to receive signals from said microphone module, said signals comprising audio, said audio comprising one or more of silent segments, cough sound segments and speech segments.

Further, the processor 201 is configured to process said received signals from said microphone module and store said processed signals in said memory 202. Processing said received signals comprise one or more of removing one or more speech components from speech segments to render the speech unintelligible and clipping said silent segments where one or more speech components include vowel sounds. Also, processing said received signals further comprises compressing said signals comprising said audio, or compressing a resultant signal after said removal of one or more speech components and/or clipping of silent segments from said signals comprising audio.

In a preferred embodiment of the invention a signal processing algorithm processes the audio signals from two microphones (two channels) to obfuscate speech within the recorded audio signals. The microphone module can comprise a non-contact microphone 203 and a contact microphone 204, suitably configured to be attached to the chest of the subject. Specific audio features are extracted from each of the two separate microphone channels to ensure that speech utterances are made unintelligible while leaving whole cough events intact. From the non-contact microphone channel, several specific audio features are extracted from the said audio signal to detect speech utterances.

Features are extracted from a number of audio frames and subsequently overlapped, for example 40 ms audio frames are overlapped by 20 ms. These features can comprise one or more of the following:

Non-contact microphone audio features (for detecting speech utterances) such as an adaptive voiced feature. The adaptive voiced feature can be defined as a measure of periodicity in the audio signal relating to the vibration of the vocal folds within a specific frequency range. For example, a frequency range of 45-500 Hz using a custom autocorrelation function can be used. A threshold value can be used, which uses values of surrounding audio frames to determine a voiced threshold value for detecting speech. A spectral centroid feature can also be used using measure of centre of mass of the frequency spectrum.

In relation to the contact microphone audio features (for detecting speech utterances) an Energy Slope feature is used in the processing. A measure of the changes in acoustic energy over time to define an energy slope. The energy slope feature is notably different when comparing speech utterances and cough events, hence, both microphone channels are employed in the detection of speech utterances.

An important aspect of the processing is the use of dual channel audio features obtained from the microphones where an energy ratio can be calculated. The energy ratio is a measure of the ratio of acoustic energy between the contact microphone and the non-contact microphone. This feature is advantageous in discriminating between cough events and third party speech.

It will be appreciated that the algorithm and features are specifically designed to detect not only adult speech, but also child speech, third party speech, and speech coming from a loudspeaker (such as a loudspeaker on a mobile phone). Loudspeaker speech has different acoustic properties compared to natural speech.

In an alternative embodiment of the invention is that the processing can be used to implement synthesising one or more speech components from speech segments to render the speech unintelligible, such that only speech utterances are synthesised from the cough audio event. In other words the invention can provide the option to “synthesise” the speech utterances rather than remove them from the said audio signals. The voiced segment of speech is synthesised by extracting specific features from the said audio frame and transforming it into a synthesised waveform.

The advantage of this approach is that the audio signal visually resembles the original audio signal however, the voiced segment of speech signals are made completely unintelligible. This approach can be useful, for example, for determining events such as sleep events from the cough audio recordings.

It can show that the subject wearing the device may be speaking (showing the subject is awake) but with sensitive information contained in the speech obfuscated. The synthetic voiced signal is generated by extracting the fundamental frequency relating to the original pitch of the voiced signal. The acoustic energy of the audio frame is also extracted.

A synthetic signal is then generated using the extracted fundamental frequency (with the first two harmonics), acoustic energy and with some random noise added to it. This synthetic signal can be constructed as:

xsynthetic(t)=A·(sin(2πf0t)+B·sin(2πf1t)+C·sin(2πf2t)+γ(t))
Where xsynthetic(t) is the generated synthetic signal
A is the amplitude of the original speech audio frame
B and C are between 0 and 1
f0 is the fundamental frequency of the original speech audio frame
f1 is the first harmonic of the original speech audio frame
f2 is the second harmonic of the original speech audio frame
γ(t) is random white noise.

The algorithm synthesises voiced frames of audio and is specifically designed to suit both human manual counting of cough events through visual and aural assessment, and automatic detection of cough events using an audio-based cough detection algorithm.

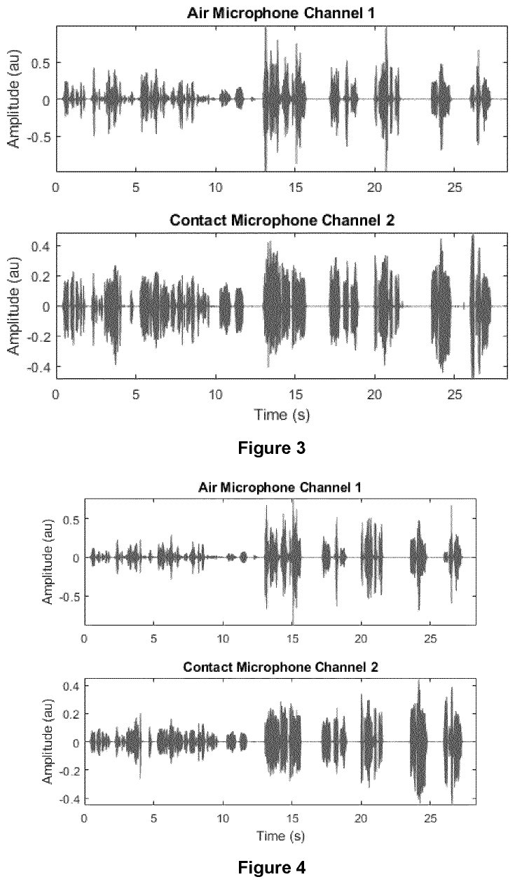

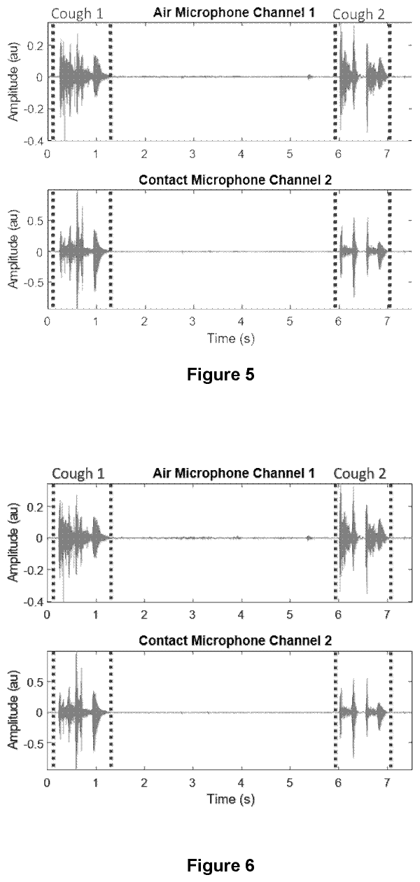

FIGS. 3-6 illustrates a number of speech and cough audio signals outputted by the algorithm according to an embodiment of the present invention showing both original and synthesised versions from the algorithm output. The speech example shown in FIGS. 3 and 4 is a snippet of a conversation between the patient wearing the device and a third party speaker on the other end of the phone where the third party speech is audible in the original file. The cough example in FIGS. 5 and 6 contains two separate cough events.

The cough monitor further comprises an accelerometer 205 operatively coupled to said processor 201 to obtain the severity of cough from said accelerometer readings, wherein said accelerometer 205 is mechanically coupled to the chest of the subject. The cough monitor further comprises a wireless transceiver 206 for transmitting said processed signals to a server.

The microphone module comprises an air microphone 203 configured to be attached to the lapel and a contact microphone 204 configured to be attached to the chest of the subject. The air microphone 203 and said contact microphone 204 are connected to the cough monitor 200 via a single connection port. In an embodiment, the microphone module comprises an air microphone 203 built into the cough monitor and a contact microphone 204 built into the cough monitor 200, said cough monitor 200 and said contact microphone 204 configured to be attached to the chest of the subject using a biodegradable adhesive.

In an embodiment, the processor 201 is configured to store the processed signals in a selected format selected from a set of predetermined formats. In an embodiment, memory 202 comprises a solid state drive, a removable secure digital card, and/or an encrypted memory encrypted with Advanced Encryption Standard 256 bit.

In an embodiment, the cough monitor 200 switches/powers on when a removable secure digital card is inserted in a secure digital card slot of the cough monitor and switches/powers off in absence thereof.

In an embodiment, the processor 201 is configured to detect one or more fault condition comprising low battery, battery door removal, faulty sensors, short circuit across sensors, open circuit across sensors, insufficient memory, memory absent, and/or clock reset or other fault condition.

The cough monitor, further comprises a user interface to allow the subject to mute said air microphone, or indicate waking time, or indicate sleeping time, or indicate medication dosing time or programmable events.

In an embodiment, the system comprises a server, where the server is configured to receive one or more audio recordings from a cough monitor via a network. The server may also receive one or more audio recordings physically on a secure digital card. The audio recordings comprising one or more of silent segments, cough sound segments and speech segments. The server is configured to process said audio recordings where processing of said received audio recordings comprise one or more of removing one or more speech components from speech segments to render the speech unintelligible and clipping said silent segments where one or more speech components include vowel sounds. Also, processing said received signals further comprises compressing said audio recordings comprising said audio, or compressing a resultant audio recording after said removal of one or more speech components and/or clipping of silent segments from said audio recordings comprising audio.

Thereby, the method and system for recording using a microphone for monitoring cough for extended periods without affecting the privacy of the subject individual or of individuals surrounding said subject individual and only requiring a fraction of duration of recorded time for a semi-skilled person to monitor cough of a subject/patient.

Further, a person ordinarily skilled in the art will appreciate that the various illustrative logical/functional blocks, modules, circuits, and process steps described in connection with the embodiments disclosed herein may be implemented as electronic hardware, or a combination of hardware and software. To clearly illustrate this interchangeability of hardware and a combination of hardware and software, various illustrative components, blocks, modules, circuits, and steps have been described above generally in terms of their functionality. Whether such functionality is implemented as hardware or a combination of hardware and software depends upon the design choice of a person ordinarily skilled in the art. Such skilled artisans may implement the described functionality in varying ways for each particular application, but such obvious design choices should not be interpreted as causing a departure from the scope of the present invention.

The process described in the present disclosure may be implemented using various means. For example, the apparatus described in the present disclosure may be implemented in hardware, firmware, software, or any combination thereof. For a hardware implementation, the processing units, or processors(s) or controller(s) may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

For a firmware and/or software implementation, software codes may be stored in a memory and executed by a processor. Memory may be implemented within the processor unit or external to the processor unit. As used herein the term “memory” refers to any type of volatile memory or non-volatile memory.

In the specification the terms “comprise, comprises, comprised and comprising” or any variation thereof and the terms include, includes, included and including” or any variation thereof are considered to be totally interchangeable and they should all be afforded the widest possible interpretation and vice versa.

A person skilled in the art would appreciate that the above invention provides a robust and economical solution to the problems identified in the prior art.

The invention is not limited to the embodiments hereinbefore described but may be varied in both construction and detail.

1. A cough monitor for a subject, comprising:
a processor;
a microphone module, having a first microphone and a second microphone, operatively coupled to the processor;
a memory operatively coupled to the processor;
said processor configured to:
receive signals from said first microphone and second microphone, said signals comprising audio, wherein the audio comprises cough sound segments and speech segments to define a cough audio event;
process said received signals from said microphone module by removing one or more speech components from speech segments to render the speech unintelligible, such that only speech utterances are removed from the cough audio event; and
store said processed signals in said memory.

2. The cough monitor of claim 1, wherein processing said received signals further comprises:
clipping said silent segments; and/or
removing extraneous non-cough noise.

3. The cough monitor of claim 1, wherein specific audio features are extracted to detect speech utterances from an audio signal received from said first microphone.

4. The cough monitor of claim 1, wherein the specific audio features extracted comprises a measure of periodicity in the audio signal relating to the vibration of the vocal folds within a specific frequency range using a custom autocorrelation function.

5. The cough monitor of claim 1, wherein said processing comprises the step of using values of surrounding audio frames to determine a voiced threshold value for detecting speech over a specific frequency range.

6. (canceled)

7. The cough monitor of claim 1, wherein processing said signals comprises measuring the changes in acoustic energy over time using an energy ratio to discriminate between speech utterances and cough events,

8. The cough monitor of claim 1, wherein an energy ratio comprises a measure of the ratio of acoustic energy between the first microphone and the second microphone to discriminate between cough events and third party speech.

9. The cough monitor of claim 1, wherein processing said received signals further comprises:
compressing said signals comprising said audio; or
compressing a resultant signal after said removal of one or more speech components and/or clipping of silent segments from said signals comprising audio.

10. The cough monitor of claim 1, wherein one or more speech components include vowel sounds.

11. The cough monitor of claim 1, further comprising an accelerometer operatively coupled to said processor to obtain the severity of cough from said accelerometer readings, wherein said accelerometer is mechanically coupled to the chest of the subject.

12. The cough monitor of claim 1, further comprising a gyroscope operatively coupled to said processor to obtain the severity of cough from said gyroscope readings.

13. The cough monitor of claim 1, further comprising a wireless transceiver for transmitting said processed signals to a server or for wireless communication with one or more sensors.

14. The cough monitor of claim 1, wherein the first microphone comprises
an air microphone configured to be attached to a lapel of the subject; and
the second microphone comprises a contact microphone configured to be attached to the chest of the subject.

15. The cough monitor of claim 14, wherein an air microphone and contact microphone are connected to the cough monitor via a single connection port or a wireless connection.

16. The cough monitor of claim 1, wherein said first microphone module comprises:
an air microphone built into the cough monitor; and said second microphone comprises
a contact microphone built into the cough monitor, said cough monitor and said contact microphone configured to be attached to the chest of the subject.

17. A method for cough monitoring, comprising the steps of:
receiving signals from a first microphone and a second microphone, said signals comprising audio, wherein the audio comprises cough sound segments and speech segments to define a cough audio event;
processing said received signals by removing one or more speech components from speech segments to render the speech unintelligible, such that only speech utterances are removed from the cough audio event; and
storing said processed signals in memory.

18. The method of claim 17, wherein processing said received signals comprise one or more of:
removing one or more speech components from speech segments to render the speech unintelligible;
clipping said silent segments; and
removing extraneous non-cough noise.

19. The method of claim 17, wherein specific audio features are extracted to detect speech utterances from an audio signal received from said first microphone.

20. The method of claim 17, wherein specific audio features extracted comprises measuring a periodicity in the audio signal relating to the vibration of the vocal folds within a specific frequency range using a custom autocorrelation function.

21. The method of claim 17 comprises the step of using values of surrounding audio frames to determine a voiced threshold value for detecting speech over a specific frequency range.

22. (canceled)

23. The method of claim 17 comprising the step of measuring the changes in acoustic energy over time using an energy ratio to discriminate between speech utterances and cough events,

24. The method of claim 17, wherein an energy ratio comprises a measure of the ratio of acoustic energy between the first microphone and the second microphone to discriminate between cough events and third party speech.

25. The method of claim 17, wherein processing said received signals further comprises:
compressing said signals comprising said audio; or
compressing a resultant signal after said removal of one or more speech components and/or clipping of silent segments from said signals comprising audio.

26. The method of claim 17, further comprising detection of one or more fault conditions, wherein the one or more conditions comprises low battery, battery door removal, faulty sensors, short circuit across sensors, open circuit across sensors, insufficient memory, memory absent, and/or clock reset.

27. The method of claim 17, further comprising monitoring the status of the module by determining the status of energy harvesting parameters during use.

28. A cough monitor for a subject, comprising:
a processor;
a microphone module, having a first microphone and a second microphone, operatively coupled to the processor;
a memory operatively coupled to the processor;
said processor configured to:
receive signals from said first microphone and second microphone, said signals comprising audio, wherein the audio comprises cough sound segments and speech segments to define a cough audio event;
process said received signals from said microphone module by synthesising one or more speech components from speech segments to render the speech unintelligible, such that only speech utterances are synthesised from the cough audio event; and
store said processed signals in said memory.
